(19) United States
(12) Patent Application Publication
(10) Pub. No.: US 2002/0179094 A1
(43) Pub. Date: Dec. 5, 2002

(54) Indicia members and method for preventing wrong-site medical procedures

(76) Inventor: David L. Perlow, Atlanta, GA (US)

Correspondence Address: EPSTEIN, EDELL, SHAPIRO, FINNAN & LYTLE, LLC, 1901 RESEARCH BOULEVARD, SUITE 400, ROCKVILLE, MD, 20850, US

(21) Appl. No.: 09/865,563

(22) Filed: May 29, 2001

(51) Int. Cl. A61B 19/00

(52) U.S. Cl. 128/897

(57) ABSTRACT

An indicia member for preventing wrong-site medical procedures includes a support for being releaseably secured on an anatomical site that is not to be subjected to a medical procedure, with the support carrying pre-printed indicia and literally communicating that the anatomical site is the wrong site for the medical procedure. The indicia may also include symbolic indicia graphically or pictorially communicating that the anatomical site is not to be subjected to a medical procedure. A method for preventing a medical procedure from being performed at the wrong anatomical site comprises the steps of releaseably securing an indicia member on an anatomical site that is not to be subjected to a medical procedure and literally, verbally communicating, via pre-printed indicia of the indicia member, that the anatomical site is the wrong site for the medical procedure.

### BACKGROUND OF THE INVENTION

1. Field of the Invention

The present invention relates generally to indicia for preventing wrong-site surgeries and other wrong-site medical procedures. More particularly, the present invention relates to indicia members for being placed at anatomical sites that are not to be subjected to medical procedures and to methods for preventing medical procedures from being performed at the wrong anatomical sites.

2. Brief Description of the Related Art

The term “wrong-site” has come to be known in the medical field in the context of medical procedures erroneously performed at the incorrect or wrong anatomical site. More commonly, “wrong-site” has been used to refer to incorrect or improper laterality where a contralateral limb or body part, e.g. left knee instead of right knee, is mistakenly subjected to a medical procedure, such as surgery. Numerous infamous cases of wrong-site surgeries have caused much attention to be focused on the need to prevent or deter surgeries from being performed on the wrong limbs or body parts. Various factors are believed to contribute to the occurrence of wrong-site surgeries including failures of communication, documentation and/or preoperative assessment; the involvement of more than one surgeon in a particular case; the need to perform multiple surgical procedures on a patient at one time, particularly on different sides of the patient's body; time pressures involving a hastening of start times and/or preoperative procedures; and unusual patient characteristics, such as physical deformity or obesity, which alter the usual processes for equipment set-up, patient preparation and/or patient positioning.

Various proposals have been set forth by the medical profession to reduce or eliminate wrong-site surgeries. One proposal calls for a surgeon to write the initials of his or her name in permanent ink on the patient's skin at the correct surgical site. During the surgical procedure, the surgeon incises through or adjacent the initials. The use of handwritten initials has various drawbacks and has failed to achieve eradication of wrong-site surgeries. In addition to the risk of infection for the patient due to ink at the surgical site, the initials may partially or completely disappear at the time of the surgical scrub. The initials may also create confusion where more than one surgical procedure is to be performed on the patient and/or where more than one surgeon is involved in the patient's treatment. In actual current clinical practice, the use of handwritten initials is infrequent. Also, because the contralateral or incorrect limb or body part is not marked in any way, it is still susceptible to being erroneously operated on. Mistakes arising from application of the initials to the wrong site are problematic to correct prior to the surgical procedure since the semi-permanence of the ink prevents the incorrectly applied initials from being completely removed. Accordingly, if the surgeon inadvertently writes his or her initials at an incorrect site and thereafter rectifies the mistake by writing his or her initials at the correct site, uncertainty and confusion necessarily result from the presence of initials at two locations on the patient's body. Even if the incorrectly applied initials are crossed out, the presence of markings at multiple anatomical sites on the patient increases the potential for human error. The correction of mistakes arising from the application of the initials to the wrong site would be facilitated through the use of removable ink. However, the use of removable or non-permanent ink would create a situation where correctly applied initials may inadvertently rub off or be removed prior to surgery. A further drawback associated with the use of initials is that the initials fail to literally communicate any definitive information about the anatomical location to which they are applied. An individual without prior knowledge cannot determine from the initials themselves what the initials are intended to designate. Accordingly, considerable institutional training is necessary to ensure that all the medical personnel involved in the surgical procedure are consistently educated as to the significance of the initials. Another drawback to handwritten initials is that conventional ink colors may not be readily visible when applied to certain skin tones so that the effectiveness of the initials may be greatly compromised.

Less preferred proposals for reducing or eliminating wrong-site surgeries include writing “NO” or “X” in black marker on the contralateral limb or body part, i.e. the side not to be operated on. These proposals have not received much endorsement due to their numerous disadvantages. The letters may be overlooked if not written large enough and/or may be inadvertently hidden when surgical drapes are applied. The “X” may rub off on another limb or body part, resulting in more than one marked area on the patient's body. The “X” is ambiguous on its face, since it may be interpreted as designating either the correct site or the incorrect site. The “NO”, when viewed upside down, reads “ON” and may have a positive interpretation. Both “NO” and “X” may be ambiguous to an individual without prior knowledge of the significance of “NO” and “X” to the anatomical site to which they are applied. Where indelible ink is utilized, inadvertent application of “NO” or “X” to the correct site is difficult to rectify without creating confusion, since application thereafter of “NO” or “X” to the incorrect site results in markings at multiple locations on the patient's body. Furthermore, markings in ink may not contrast sufficiently with certain skin tones and may go unnoticed. Other less preferred proposals to prevent wrong-site surgeries include adhering a yellow sticky dot above an eye to be operated on and applying a Band-Aid over an eye to be operated on. The use of sticky dots or Band-Aids, however, creates uncertainty with regard to the meaning or significance intended from their presence.

U.S. Pat. No. 4,947,867 to Keeton discloses a label for being adhered to a patient's garb or skin and including blanks for information to be written in regarding the patient's name, procedure, surgeon's name, date and patient signature. There is no way to ensure that the person filling in the “procedure” blank will specifically identify the correct anatomical site for the procedure. Accordingly, Keeton proposes the use of an arrow on the label or on a separate label to indicate the correct site for the procedure. Contralateral limbs or body parts remain unmarked in any way and are thusly at risk of being operated on inadvertently. There is nothing to prevent an incorrect site from being prepared for surgery and operated on erroneously while the label for the correct site goes undetected. Moreover, the label proposed by Keeton is inherently inaccurate. It can not be placed directly on or over the correct site since the arrow, by nature, points to a distant or remote location. The arrow gives rise to guesswork because the actual location of the correct site in relation to the arrow tip is not known.

Adhesive labels or adhesive members for being applied to a person's body for medical purposes are shown in U.S. Pat. No. 4,860,331 to Williams et al, U.S. Pat. No. 5,306,271 to Zinnreich et al, U.S. Pat. No. 5,384,174 to Ward et al, and U.S. Pat. No. 5,407,440 to Zinnreich et al. The Zinnreich et al patents relate to skin markers used to delineate a radiation therapy portal area. The Ward et al patent relates to an adhesive sheet material for use on the skin as a wound dressing, a dressing for a catheter or an ostomy flange. The patent to Williams et al relates to tape for being placed on a patient's skin to provide visual reference points on an image of the patient's body obtained by a scanning procedure.

It can be seen from the above that presently proposed indicia and indicia members placed on a patient's body for the purpose of identifying the correct site for a medical procedure fail to protect against the medical procedure being performed at an incorrect site and have not eradicated the occurrence of wrong-site surgeries. It is also apparent from the above that the indicia thus far proposed for being placed on a patient's body for the purpose of identifying the incorrect site for a medical procedure have numerous drawbacks and have not been widely accepted. The various disadvantages associated with presently proposed indicia and indicia members have resulted in the absence of a uniformly accepted standard for preventing wrong-site surgeries. The use of different indicia and/or indicia members among physicians and other medical personnel and hospitals is undesirable and leads to confusion, uncertainty and an increased risk for human error. Accordingly, there is a great need for a standardized indicia member carrying clearly unambiguous indicia and which can be placed at an anatomical site to identify the anatomical site, with certainty, as the incorrect site for a medical procedure. While presently proposed indicia and indicia members are designed for use by the medical profession, it would be desirable to empower patients along with medical professionals by providing an indicia member carrying clearly unambiguous indicia that a patient or medical professional can apply to an anatomical site to effectively ensure that the anatomical site is not subjected to a medical procedure in error.

### SUMMARY OF THE INVENTION

Accordingly, it is a primary object of the present invention to overcome the aforementioned disadvantages of prior indicia and indicia members proposed to eliminate wrongsite medical procedures.

Another object of the present invention is to prevent an anatomical site from being subjected to a medical procedure in error by providing an indicia member at the anatomical site including one or more printed words having an unambiguous literal meaning.

A further object of the present invention is to identify an anatomical site as the wrong site for a medical procedure via an indicia member applied to the anatomical site and including verbal indicia literally communicating that the anatomical site is the wrong site for the medical procedure.

Additionally, it is an object of the present invention to use both verbal and symbolic indicia on an anatomical site to indicate redundantly that the anatomical site is not to be subjected to a medical procedure.

Yet another object of the present invention is to provide standardized pre-printed indicia for being applied by medical personnel and/or patients to anatomical sites that are not to be subjected to medical procedures.

The present invention has as a further object to permit indicia members of various external sizes to be obtained from an elongate strip of material, with the indicia members each carrying indicia communicating that a medical procedure is not to be performed.

A still further object of the present invention is to circumscribe an anatomical body part with an indicia member carrying indicia communicating that the body part is not to be subjected to a medical procedure.

Some of the advantages of the present invention are that the indicia members are conspicuous against all skin tones; the indicia members are highly unlikely to be overlooked or draped inadvertently; the indicia members may incorporate various and/or multiple colors to enhance noticeability; the symbolic indicia may include readily understood pictures or symbols; the indicia members can be placed directly on or over the incorrect anatomical sites; the correct anatomical sites are not exposed to inks, adhesives or other extraneous substances; the indicia members can be secured to the incorrect anatomical sites with an adhesive strong enough to ensure that the indicia members can not become inadvertently detached from the incorrect anatomical sites while allowing the indicia members to be removed without significant discomfort for patients after the medical procedures have been successfully performed at the correct anatomical sites; the indicia are not susceptible to varying interpretations and multiple meanings; the indicia do not require inferences or background knowledge to be understood; indicia members may be applied to multiple incorrect anatomical sites on a patient without creating confusion or uncertainty, even in cases where multiple medical procedures are to be performed on the patient at one time; patients may experience less anxiety due to the enhanced clarity and certainty that the indicia members provide; and the indicia members can be used on various body parts of humans and animals.

These and other objects, advantages and benefits are realized with the present invention as generally characterized in an indicia member including a support for being releaseably secured on an anatomical site that is not to be subjected to a medical procedure and carrying indicia including verbal indicia literally communicating that the anatomical site is the incorrect site for the medical procedure. The verbal indicia includes one or more words, preferably “wrong-site” and/or “stop”, of unambiguous and definitive significance. The indicia may also include symbolic indicia graphically or pictorially communicating that the anatomical site is not to be subjected to a medical procedure. The verbal and symbolic indicia provide redundant protection against error and may be arranged on the support in a manner to optimize clarity of meaning. The support may include a sheet of material on which the indicia is pre-printed and a layer of adhesive disposed on the sheet of material, with or without a removable backing sheet provided over the layer of adhesive, for releaseably securing the sheet of material on the anatomical site. The indicia member may be obtained from an indicia device including an elongate strip or tape carrying the indicia thereon in a repeating pattern. A selected length of the strip or tape can be separated or detached from the remainder thereof to obtain an indicia member of desired external size. The length of the strip or tape selected for separation or detachment may be sufficiently long to permit the resulting indicia member to circumscribe or encircle an anatomical body part or limb. The strip or tape may be wound into a roll.

The present invention is also generally characterized in a method for preventing a medical procedure from being performed at the wrong anatomical site comprising the steps of releaseably securing a sheet of material having pre-printed indicia thereon on an anatomical site that is not to be subjected to a medical procedure and literally, verbally communicating, via the indicia, that the anatomical site is the wrong site for the medical procedure. The method may include the step of pictorially communicating via the indicia that the anatomical site is not to be subjected to the surgical procedure.

The above and still further objects, features and advantages of the present invention will become apparent upon consideration of the following detailed description of preferred embodiments thereof, particularly when taken in conjunction with the accompanying drawings, wherein like reference numerals in the various figures are utilized to designate like components.

### BRIEF DESCRIPTION OF THE DRAWINGS

FIG. 1 is a front view of an indicia member according to the present invention.

FIG. 2 is a perspective view showing an adhesive layer of the indicia member.

FIG. 3 is a perspective view of an indicia device comprising an elongate strip or tape wound into a roll and carrying end-facing indicia in a repeating pattern.

FIG. 4 is a perspective view of an alternative indicia device comprising an elongate strip or tape wound into a roll and carrying side-facing indicia in a repeating pattern.

FIG. 5 is a front perspective view of a human body illustrating an indicia member applied to a knee that is not to be subjected to a medical procedure.

FIG. 6 is a broken, front perspective view of a human knee showing an indicia member obtained from the continuous strip or tape of FIG. 4 circumscribing or encircling the knee.

### DESCRIPTION OF THE PREFERRED EMBODIMENTS

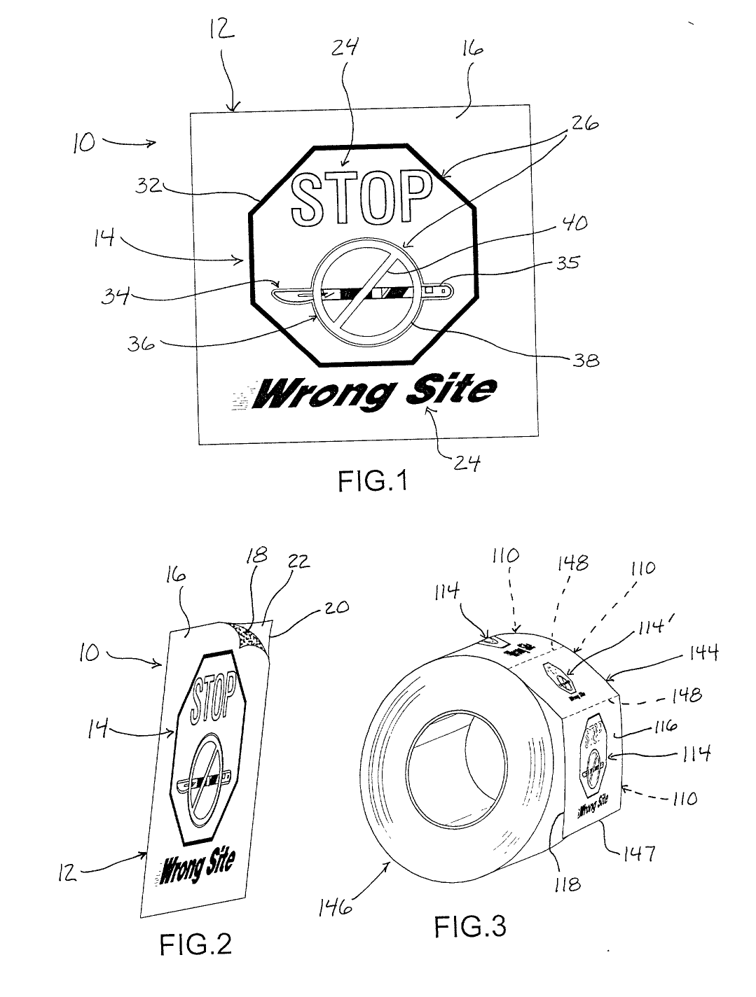

An indicia member 10 according to the present invention is illustrated in FIGS. 1 and 2 and includes an indicia-carrying element or support 12 and indicia 14 carried by 12. The support 12 is adapted to be releaseably secured on a patient's skin at an incorrect or wrong anatomical site for a medical procedure, i.e. an anatomical site, body part or limb that is not to be subjected to a medical procedure. The support 12 is illustrated as an adhesive label, but can be designed in various other ways to carry indicia and be releaseably secured on the patient's body. The support 12 includes a sheet of material 16 having a forward face carrying the indicia 14 and having a rearward face carrying an adhesive layer 18. Optionally, a backing or cover sheet 20 may be disposed over adhesive layer 18 as shown in FIG. 2. The sheet 16 can be of any suitable material, such as foam, paper or plastic capable of carrying indicia 14 and can have any suitable external configuration, such as rectangular, square, etc., of any suitable size, for example 2½ inches by 2½ inches. The sheet 16 is preferably flexible and of minimal thickness to conform to the shape of the anatomical site, body part or limb to which it is applied and to facilitate an elongate strip or tape formed by sheet 16 being wound into a roll as explained further below. The use of a foam material for sheet 16 is preferred, and provides the benefits of comfort to the wearer and ease of use.

The adhesive layer 18 is typically coextensive with the entire surface area of the rearward face of sheet 16, but may be disposed on less than the entire surface area of the rearward face. As an example, the layer 18 may be disposed on one or more portions of the surface area of the rearward face less than the entirety thereof. Where the layer 18 is disposed on more than one portion of the surface area, the portions may be discrete or may be contiguous or touching. The adhesive layer 18 can be of any suitable adhesive safe for use on the skin. Preferably, the adhesive layer 18 is made of a pressure-sensitive adhesive having an adhesive strength to releaseably secure sheet 16 to the patient's skin with sufficient force to prevent it from becoming inadvertently detached from the skin, while still allowing the sheet 16 to be deliberately removed without causing the patient significant discomfort. It should be appreciated that the sheet 16 can be releaseably secured on the patient in various ways in addition to or as an alternative to adhesive. For example, the sheet 12 can be provided or formed with bands or straps made of elastic material and/or having opposing ends with fasteners by which the bands or straps can be secured around a body part.

The backing sheet 20, where provided, may be disposed over the adhesive layer 18 coextensive therewith and has a forward face 22 releaseably adhered to the adhesive layer 18. The backing sheet 20 may comprise a single sheet of material coextensive with the entire surface area of the rearward face of sheet 16. However, the backing sheet 20 may comprise a plurality of backing sheet segments or portions and/or may be disposed over less than the entire surface area of the rearward face, such as where the adhesive layer 18 is disposed over less than the entire surface area of the rearward face. At least the forward face 22 of backing sheet 20 is made of a suitable material capable of adhering to the adhesive layer 18 while allowing the backing sheet to be released from the adhesive layer with minimal force when the backing sheet is manually peeled away or removed from sheet 16 as shown in FIG. 2. The backing sheet 20 can be provided with grasping structure such a tab, flap or other structure to facilitate grasping of the backing sheet for removal from sheet 16. The backing sheet 20, where provided, is disposed over the adhesive layer 18 prior to use of the indicia member 10 to protect the adhesive layer and control unwanted adherence thereof to inadvertent objects or surfaces. When it is desired to use the indicia member 10, the backing sheet 20 is manually grasped, typically at a corner or at grasping structure provided for such purpose, and is peeled away from the adhesive layer 18 and sheet 16. In this manner, the backing sheet is removed entirely from the adhesive layer 18 and sheet 16, thusly exposing the adhesive layer. The sheet 16 is then ready to be secured on the patient's body via the now exposed adhesive layer.

Indicia 14 comprises printed indicia including verbal indicia 24 and symbolic indicia 26. Preferably, indicia 14 is pre-printed on sheet 16, such as by machine printing, and is permanent or indelible. What is meant by “pre-printed” is that the indicia 14 is printed on sheet 16 prior to the indicia member being supplied for use so that no information needs to be hand-written in by the user. Also, the indicia 14 may be the same for all patients and medical procedures. Verbal indicia 24 includes one or more words literally communicating in unambiguous language that an anatomical site to which the indicia member 10 is applied is the incorrect or wrong site for a medical procedure. In a preferred embodiment, the verbal indicia 24 includes the verbal statements “wrong-site” and/or “stop”. In the illustrated embodiment, the verbal indicia 24 includes both “wrong-site” and “stop” for redundant protection against error. Although various alternative language and numerous synonyms for “wrong-site” and “stop” could be used, the terms “wrongsite” and “stop” are preferred for their clarity in that the unambiguous literal meanings for “wrong” and “stop” are commonly known and are not subject to interpretation or inference. In addition, the term “wrong-site” has acquired a specific meaning in the medical profession. It is virtually impossible for anyone to not understand the message communicated literally by the verbal indicia 24. It should be appreciated that the verbal indicia 24 can be modified for use of indicia member 10 in foreign countries where English may not be understood. Accordingly, foreign words similar in meaning and clarity to “wrong-site” and/or “stop” may be used.

Symbolic indicia 26 includes one or more unambiguous symbols communicating graphically, pictorially or non-verbally that an anatomical site to which the indicia member 10 is applied is the incorrect or wrong site for a medical procedure. In the preferred embodiment, the symbolic indicia 26 includes an octagon 32, preferably red in color, containing the verbal indicia “stop” in capital letters in the manner of a conventional “STOP” sign. The use of such a universally recognized symbol eliminates ambiguity and further reinforces the literal meaning communicated by the verbal indicia. Symbolic indicia 26 further includes a pictorial representation 34 comprising a picture or illustration 35 of a medical instrument, such as a scalpel, and a graphical sign 36 superimposed over illustration 35. The graphical sign 36 negates the illustration 35 and includes a circular ring 38 and a diagonal line 40 bisecting the circle circumscribed by ring 38 diametrically. Together, illustration 35 and graphical sign 36 communicate unambiguously that a medical procedure is not to be conducted. Even further clarity is realized from the arrangement of pictorial representation 34 superimposed over and disposed within the octagon 32, beneath the term “stop”. With this arrangement, the octagon 32, the word “stop”, and the pictorial representation 34 clearly warn against the performance of a medical procedure.

The verbal indicia 24 and the symbolic indicia 26 are arranged on the forward face of sheet 16 with the terms “wrong-site” centered beneath and disposed externally of octagon 32, which contains the word “STOP” and pictorial representation 34 centered beneath the word “STOP”. An individual indicia member 10 may include one or more indicia 14 on the forward face of sheet 16 and, where more than one indicia 14 is provided, the indicia 14 can be the same size or different sizes. An indicia member having more than one indicia 14 can be cut or severed, such as with scissors, to form a plurality of smaller size indicia members each having at least one indicia 14.

The forward face of sheet 16 is preferably rendered in colors so that the indicia 14 contrast conspicuously with all skin tones. In the preferred embodiment, the forward face of sheet 16 has a white background with the words “wrong-site” being depicted in black. The octagon 32 is red with a black outline, and the term “stop” is formed of white letters outlined in black. The ring 38 is set within a white circle. The illustration 35 is rendered in black against white with a white border where the illustration 35 is superimposed over octagon 32. It should be appreciated that the colors set forth herein are not exclusive and that many various colors can be used singly or in combination.

FIG. 3 illustrates an indicia device wherein sheet 116 is formed as an elongate strip or tape 144 wound into a roll 146. The strip or tape 144, which may be provided with or without a backing sheet, has a free end 147 for grasping. The strip or tape 144 is continuous between end 147 and an opposite, terminal end (not visible in FIG. 3). A layer of adhesive 118 is disposed on the rearward face of strip or tape 144, and a plurality of indicia are arranged on the forward face of strip or tape 144 in a linearly repeating pattern. In the illustrated embodiment, this pattern includes alternating indicia 114 and 114′ with indicia 114 being of a first size and indicia 114′ being of a second size, smaller than the first size. FIG. 3 illustrates one manner of arranging indicia 114 and 114′ on strip or tape 144, although the arrangement depicted in FIG. 3 is less preferred than that depicted in FIG. 4. The indicia 114 and 114′ face end 147 and are right side up when viewed from end 147. Accordingly, indicia 114 and 114′, which may face either end of strip or tape 144, may be considered end-facing indicia. A selected length segment of strip or tape 144 may be ? separated or detached from the remainder thereof, with the separated or detached length segment defining an indicia member. The resulting indicia member will have at least one indicia 114 or 114′, but may have more than one indicia depending on the size, i.e. length, of the separated or detached length segment. The strip or tape 144 may optionally be provided with perforated lines, score lines or any other pre-formed frangible junctures 148 , shown in dotted lines in FIG. 3, facilitating separation of length segments from the remainder of strip or tape 144. The junctures 148 may be disposed at predetermined intervals or a predetermined spacing along strip or tape 144 to define a plurality of serially interconnected, pre-formed indicia members 110 as shown by dotted lines in FIG. 3. Of course, the indicia 114, 114′ and/or the junctures 148 can be located so that the indicia members 110 are the same size or different sizes. It should be appreciated that roll 146 can be mounted in a suitable tape dispenser which may include a cutting member, such as a serrated edge, by which a selected length segment of the strip or tape may be separated from the remainder thereof. It should be further appreciated that strip or tape 144 can be provided without frangible junctures in that a selected length segment of strip or tape 144 may be cut from the remainder thereof with scissors or with the cutting member of a dispenser or may be detached by tearing as described below for strip or tape 244.

FIG. 4 illustrates a preferred embodiment of an indicia device including an elongate strip or tape 244 wound into a roll 246. The strip or tape 244 is similar to strip or tape 144 but does not have any frangible junctures and carries side-facing indicia 214 and 214′ in a repeating pattern. Indicia 214 and 214′ alternate and face laterally or perpendicular to free end 247. A selected length segment of strip or tape 244 can be separated or detached, such as by cutting with scissors or the cutting member of a tape dispenser or by tearing, from the reminder thereof to obtain an indicia member of desired external size.

In use, an indicia member is releaseably secured to an anatomical site, typically a contralateral limb or body part, that is not to be subjected to a medical procedure. After the correct site for the medical procedure has been verified, the indicia member is secured on the patient's body at a location corresponding to but contralateral to the correct site. FIG. 5 illustrates indicia member 10 releaseably secured to the left knee LK of a patient whose right knee RK is to be subjected to a surgical procedure. The indicia member 10 is secured on the left knee LK via contact of adhesive layer 18 with the patient's skin over the left knee. Since the actual surgical procedure may be conducted at an internal operative site within the patient's body, such as within the right knee RK, the correct site may be considered as the external site at which entry or access to the internal operative site is gained via an artificially or naturally created opening such as an incision, an endoscopic portal, an anatomical passage or the like. In the example illustrated in FIG. 5, the correct site for the surgical procedure is on the anterior of the right knee RK; and, accordingly, the indicia member 10 is secured on the anterior of the left knee LK. As a further example, if an internal operative site within the right knee were to be accessed from the posterior of the right knee, the indicia member 10 would be placed on the posterior of the left knee.

The indicia member 10 can be placed directly at, on or over the incorrect site such that it can not be inadvertently overlooked or draped from view in the event that the incorrect site is mistakenly approached for surgery. With the indicia member secured in place at the incorrect site, the incorrect site can not be operated on erroneously. Medical personnel attempting to prepare the incorrect site, i.e., the left knee in FIG. 5, for surgery and/or to operate on the incorrect site in error necessarily encounter the indicia member 10. Upon encountering indicia member 10, medical personnel are unequivocally alerted to the fact that the left knee is not the correct site for the surgical procedure, thereby thwarting mistakes before they can occur. After the medical procedure has been performed at the correct site, the indicia member 10 is manually detached from the patient's skin and disposed of.

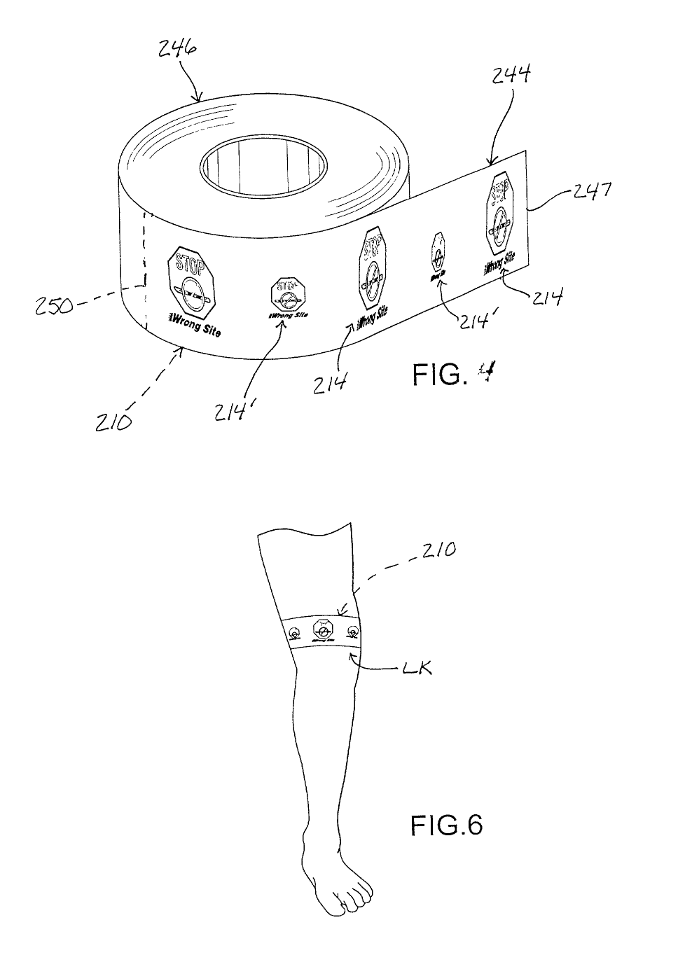

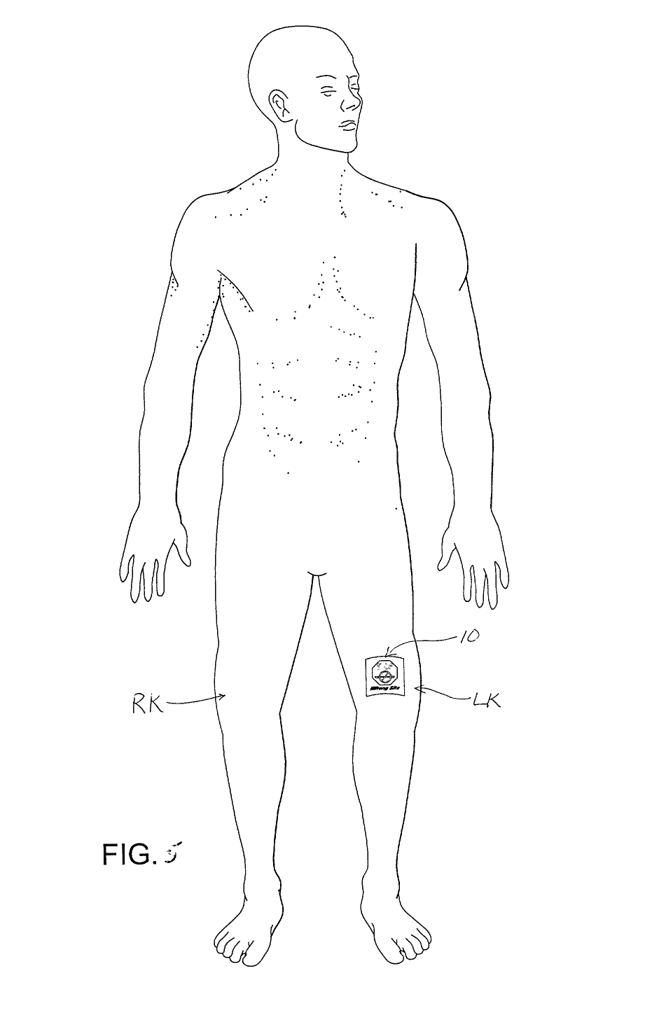

FIG. 6 is representative of a procedure wherein a length segment of strip or tape 244 sufficient in length to circumscribe or encircle a contralateral limb or body part is cut away from the remainder thereof, as indicated by cutting line 250 shown in dotted lines in FIG. 4. The resulting indicia member 210 is of sufficient length to be wrapped around the contralateral limb or body part, i.e. the left knee LK in FIG. 6, with opposite ends of the indicia member 210 overlapping or in abutment. Accordingly, an optimal size indicia member can be obtained from roll 246 in accordance with the size and/or shape of the anatomical site to which the indicia member is to be applied and/or the desired mode of attachment or securement of the indicia member at the anatomical site.

With the present invention, medical personnel are not burdened with having to search for and confirm the presence of indicia at the correct site on the patient before proceeding with the medical procedure. In addition, users of the indicia members do not have to supply any handwritten information, since all essential information is already pre-printed on the indicia members. Since the correct site remains unaltered in any way, the increased risks of infection and complications for the patient from foreign substances or materials are avoided. Indicia members may be secured at multiple incorrect sites on an individual patient without causing confusion since the message communicated literally and symbolically by the indicia members is the same for each site to which the indicia members are applied. The message communicated by the indicia members is self-evident without the need for background knowledge. Redundant levels of communication and protection against error are provided in an individual indicia member through multiple verbal indicia, multiple symbolic indicia and the presence of both verbal and symbolic indicia. The indicia cannot inadvertently rub off or become transferred to another part of the patient's body. Various sizes of indicia members can be obtained from a single strip or tape by detaching a desired size indicia member therefrom via cutting, tearing or any suitable separation procedure. The ability to vary the sizes of the indicia members during use allows the optimal size indicia member to be used for a particular anatomical site and/or desired mode of securement. The indicia members may be standardized so that they are the same for all surgeons, medical facilities, medical procedures and patients. The indicia members may be used by both medical personnel and patients undergoing medical procedures and may desirably impart feelings of empowerment to patients. Patients on whom the indicia members are used may experience greater confidence and less anxiety about their medical procedures. The indicia members can be used on various body parts not limited to contralateral limbs or body parts.

In as much as the present invention is subject to many variations, modifications and changes in detail, it is intended that all subject matter discussed above or shown in the accompanying drawings be interpreted as illustrative only and not be taken in a limiting sense.

What is claimed is:

1. An indicia member for indicating that an anatomical site on a patient is not to be subjected to a medical procedure comprising
a sheet of material for being releaseably secured on a patient's body at an anatomical site that is not to be subjected to a medical procedure, said sheet of material having a forward face and a rearward face;
indicia pre-printed on said forward face and including one or more words literally communicating that the anatomical site is the incorrect site for the medical procedure; and
a layer of adhesive on said rearward face for releasably securing said sheet of material on the patient's body.

2. An indicia member as recited in claim 1 wherein said one or more words include the words “wrong-site”.

3. An indicia member as recited in claim 1 wherein said one or more words include the word “stop” in capital letters.

4. An indicia member as recited in claim 3 wherein said one or more words include the words “wrong-site”.

5. An indicia member as recited in claim 1 wherein said indicia further includes one or more symbols graphically communicating that the anatomical site is the incorrect site for the medical procedure.

6. An indicia member as recited in claim 5 wherein said one or more symbols include a red octagon.

7. An indicia member as recited in claim 6 wherein said verbal indicia includes the word “stop” in capital letters superimposed over said octagon.

8. An indicia member as recited in claim 5 wherein said one or more symbols include a pictorial representation.

9. An indicia member as recited in claim 8 wherein said pictorial representation includes an illustration of a surgical instrument and a graphical sign superimposed over and negating said illustration.

10. An indicia member as recited in claim 9 wherein said illustration of a surgical instrument includes an illustration of a scalpel.

11. An indicia member as recited in claim 9 wherein said graphical sign includes a ring circumscribing a circle and a line bisecting said circle diametrically.

12. An indicia member as recited in claim 1 and further including a removable backing sheet disposed over said layer of adhesive.

13. An indicia member as recited in claim 1 wherein said sheet of material is made of foam.

14. An indicia device providing an indicia member for indicating that an anatomical site on a patient is not to be subjected to a medical procedure comprising
an elongate strip of material having a forward face, a rearward face and a plurality of indicia pre-printed on said forward face in a linearly repeating pattern, said indicia each communicating that a medical procedure is not to be performed, said strip of material being separable to allow a length segment thereof having at least one of said indicia thereon to be separated from the remainder of said strip of material for use as an indicia member; and
a layer of adhesive disposed on said rearward face for releaseably securing said indicia member to a patient's skin on a body part that is contralateral to a body part that is to be subjected to a medical procedure.

15. An indicia device as recited in claim 14 wherein said indicia each includes verbal indicia literally communicating that the contralateral body part is not to be subjected to a medical procedure.

16. An indicia device as recited in claim 15 wherein said indicia each includes symbolic indicia including a red octagon and said verbal indicia includes the word “stop” in capital letters within said octagon.

17. An indicia device as recited in claim 15 wherein said verbal indicia includes the words “wrong-site”.

18. An indicia device as recited in claim 16 wherein said verbal indicia further includes the words “wrong-site” disposed externally of said octagon.

19. An indicia device as recited in claim 18 wherein said symbolic indicia further includes an illustration of a surgical instrument superimposed over said octagon and a graphical sign superimposed over and negating said illustration.

20. An indicia device as recited in claim 14 wherein said strip of material is made of a foam material.

21. An indicia member for indicating that an anatomical body part is not to be subjected to a medical procedure comprising
support means for being releaseably secured on an anatomical body part that is not to be subjected to a medical procedure; and
indicia means carried by said support means for literally and pictorially communicating that the anatomical body part is the wrong site for a medical procedure.

22. A method for preventing a medical procedure from being performed at the wrong anatomical site comprising the steps of
releaseably securing a sheet of material having pre-printed indicia thereon on the body of a patient at an anatomical site that is not to be subjected to a medical procedure; and
literally, verbally communicating, via the pre-printed indicia, that the anatomical site is the wrong site for a medical procedure.

23. A method for preventing a medical procedure from being performed at the wrong anatomical site as recited in claim 22 wherein said step of securing includes securing the sheet of material on a body part of the patient that is contralateral to a body part of the patient that is to be subjected to a medical procedure.

24. A method for preventing a medical procedure from being performed at the wrong anatomical site as recited in claim 23 wherein said step of securing includes adhesively securing the sheet of material on the patient's skin.

25. A method for preventing a medical procedure from being performed at the wrong anatomical site as recited in claim 22 and further including the step of pictorially communicating, via the pre-printed indicia, that the anatomical site is not to be subjected to a medical procedure.

26. A method for preventing a medical procedure from being performed at the wrong anatomical site as recited in claim 22 and further including, prior to said step of securing, the steps of detaching a selected length segment from the remainder of an elongate strip of material having a plurality of the indicia pre-printed thereon in a linearly repeating pattern and using the detached length segment as the sheet of material.

27. A method for preventing a medical procedure from being performed at the wrong anatomical site as recited in claim 26 wherein said step of detaching includes detaching a length segment of sufficient length to encircle a body part on which the anatomical site is located and said step of releasably securing includes wrapping the detached length segment around the body part.
